(19) United States
(12) Patent Application Publication
(10) Pub. No.: US 2025/0073385 A1
(43) Pub. Date: Mar. 6, 2025

(54) MEANS OF GRAVITY IV FLOW SENSING VIA MASS CHANGE SENSOR AND DROP COUNTING DEVICE

(71) Applicant: CareFusion 303, Inc., San Diego, CA (US)

(72) Inventors: Jason Andrew WINE, Brea, CA (US); Ryan CALLAHAN, Long Beach, CA (US); Siddarth SHEVGOOR, Mission Viejo, CA (US)

(21) Appl. No.: 18/816,730

(22) Filed: Aug. 27, 2024

Related U.S. Application Data
(60) Provisional application No. 63/579,361, filed on Aug. 29, 2023.

(51) Int. Cl. A61M 5/14; A61M 5/168; A61M 5/36

(52) U.S. Cl. CPC A61M 5/1411; A61M 5/16831; A61M 5/365; A61M 2005/1401; A61M 2202/0007; A61M 2202/04; A61M 2205/15; A61M 2205/3306; A61M 2205/3327; A61M 2205/3334; A61M 2205/3379; A61M 2205/502

(57) ABSTRACT

Infusion control systems are disclosed that can control and monitor medical fluid administration for gravity-based infusion of medical fluid, where the gravity infusion control system is couplable with an intravenous administration set and can include a mass change sensor and drop counter.

### CROSS REFERENCE TO RELATED APPLICATION

This application claims the benefit of priority under 35 U.S.C. § 119 to U.S. Provisional Patent Application Ser. No. 63/579,361, entitled “MEANS OF GRAVITY IV FLOW SENSING VIA MASS CHANGE SENSOR AND DROP COUNTING DEVICE”, filed on Aug. 29, 2023, the entire contents of which is incorporated herein by reference.

### BACKGROUND

The present disclosure relates generally to administration of medical fluid, such as intravenous (“IV”) medical fluid therapy, and more particularly, to devices, systems, and methods, for controlling and monitoring gravity-based administration of medical fluid. Flow sensing features, such as a mass change sensor used in combination with a drop counting device allows for higher precision flow rates to be achieved and a broader range of control (e.g., approximately 2 mL/hr to over 15,000 mL/hr).

IV fluid delivery systems are used to deliver or infuse medical fluid to patients at controlled rates. Several such IV fluid delivery systems exist, including gravity-based systems that utilize the pressure of gravity to direct a medical fluid from an IV bag to a patient though tubing, and pump-based systems that use a mechanical pump that can engage tubing to direct a medical fluid from an IV bag to a patient.

Gravity-based infusion systems can include an IV administration set, which includes an IV bag, a drip chamber, infusion tubing, and a flow control device. The flow rate of the medical fluid to the patient is controlled by a flow control device, such as a roller clamp, a pinch clamp, or a flow valve. Generally, the patient would receive the entire volume of medical fluid from the IV bag unless the flow is interrupted, such as by the patient or a caregiver obstructing the infusion tubing or disconnecting the infusion tubing from the patient. Pump-based infusion systems include similar components as a gravity-based infusion system; however, the flow rate and volume of medicament directed to the patient can be controlled using the infusion pump instead of a flow control device as used in gravity-based infusion systems.

### SUMMARY

Although a pump-based infusion systems may provide the ability to control and monitor the administration of medical fluid, such as by controlling the fluid flow rate and volume of medical fluid directed to a patient, pump-based infusion systems can be more complicated and more expensive, relative to gravity-based infusion systems. Further, gravity and pump-based infusion systems each require a caregiver or user to assemble and configure the system, including ensuring each component is prepared and appropriately connected together, and ensuring that infusion and fluid flow parameters are set as intended. Some systems, such as gravity-based infusion systems, may require the caregiver or user to prime the fluid pathway, and maintain and/or adjust the fluid flow parameters throughout the infusion. Furthermore, some infusion systems may not provide notification if a complication or error is encountered, such as air in the fluid pathway or unintended disconnection of the system from the patient.

In accordance with at least some embodiments disclosed herein is the realization that, although gravity-based infusion systems may provide a lower cost alternative to pump-based infusions systems, pump and gravity-based infusion systems require a caregiver to set up, maintain, and monitor several aspects of the medical fluid administration. As such, the use of gravity-based infusion systems may expose a patient to several potential complications and errors, which may result in injury or insufficient care for a patient.

Accordingly, the present disclosure addresses operational challenges encountered in prior systems and methods for administering medical fluids using gravity-based infusion systems and provides a means for controlling and monitoring medical fluid administration, which can provide patient protection from complications during infusion therapy, improve workflow for clinicians and caregivers, maintain patient mobility, reduce medication administration errors, increase medication security, increase medication delivery accuracy and rate stability, lower costs, and increase documentation efficiency, as well as preventing, detecting, and resolving complications such as, an occlusion, extravasation, and air in the fluid pathway.

Features of the present embodiments can provide gravity infusion control systems that can permit control of aspects including, but not limited to, any of fluid flow rate, dosage volume, priming of the IV administration set, and automatically stopping and/or starting infusion. Further, aspects of the present disclosure can provide monitoring of aspects of the fluid pathway of the IV administration set including, but not limited to, pressure, fluid flow rate, occlusions, the presence of air, fluid flow rate, leakage, infiltration, and the potential occurrence of tampering to the IV administration set.

Embodiments of the present disclosure provide a gravity infusion control system comprising a drip chamber containing an infusate configured to be fluidly coupled to a patient by IV tubing extending therebetween, a mass change sensor configured to detect any of a change in weight or movement of the infusate in the drip chamber and a drop detector configured to detect drops of the infusate falling from the drip chamber. In some embodiments, the gravity infusion control system further comprises a user interface that controls a flow rate of the gravity infusion control system. In some embodiments, the mass change sensor is a load cell or a weight. In some embodiments, the drip chamber is coupled to the mass change sensor by an arm, a hook, or a clamp. In some embodiments, an infusate fluid flow is directed through the IV tubing by a pressure of gravity. In some embodiments, the drop detector is an infrared light emitter and receiver. In some embodiments, the infusate fluid flow is configured to provide a flow rate about 50 mL/hour, about 100 mL/hour, about 150 mL/hour, about 200 mL/hour, about 250 mL/hour, about 300 mL/hour, about 350 ml/hour, about 400 mL/hour, about 450 mL/hour, or 500 mL/hour. In some embodiments, the infusate fluid flow is configured to provide a flow rate about 2 mL/hr, about 5 mL/hr, about 10 mL/hr, about 15 mL/hr, about 20 mL/hr, about 25 mL/hr, about 30 mL/hr, about 35 mL/hr, about 40 mL/hr, about 55 mL/hr, or about 5 mL/hr. In some embodiments, the infusate fluid flow is greater than 500 mL/hr. In some embodiments, the gravity infusion control system further comprises one or more of an air in line detector, a leak detector, an external pressure module, a tamper resistance module, an information and control module, and a connectivity module.

Embodiments of the present disclosure provide a gravity infusion control system comprising an intravenous administration set coupled to a medical fluid reservoir and a flow sensor coupled to the intravenous administration set, the gravity infusion control system configured such that the flow sensor can detect any change in mass or movement of the medical fluid reservoir. In some embodiments, the flow sensor comprises a mass change sensor and a drop detector. In some embodiments, the flow sensor is configured to detect any of a change in weight or movement of the medical fluid reservoir of the intravenous administration set. In some embodiments, the mass change sensor is a load cell or a weight. In some embodiments, the drop detector is an infrared light emitter and receiver.

Embodiments of the present disclosure provide a method for providing a gravity infusion control system comprising coupling a medical fluid reservoir of an intravenous administration set to a flow detection interface, wherein the flow detection interface comprises a flow sensor configured to detect any of a change in mass or movement of the medical fluid reservoir, selecting, using a processor, a flow rate through the intravenous administration set, wherein the processor is coupled with the flow detection interface and a flow control interface, such that the processor receives data from the flow sensor to determine the presence of a fluid flow through a tubing of the intravenous administration set. In some embodiments, the flow rate selected is about 50 mL/hour, about 100 ml/hour, about 150 ml/hour, about 200 mL/hour, about 250 mL/hour, about 300 mL/hour, about 350 mL/hour, about 400 mL/hour, about 450 ml/hour, or 500 mL/hour. In some embodiments, the flow rate selected is about 2 mL/hr, about 5 mL/hr, about 10 mL/hr, about 15 mL/hr, about 20 mL/hr, about 25 mL/hr, about 30 mL/hr, about 35 mL/hr, about 40 mL/hr, about 55 mL/hr, or about 5 mL/hr. In some embodiments, the flow rate selected is greater than 500 mL/hr. In some embodiments, the flow sensor comprises a mass change sensor and a drop detector.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates a gravity infusion control system in use with an IV administration set coupled to a patient, in accordance with aspects of the present disclosure.

FIG. 2 illustrates a gravity infusion control system in use with an IV administration set including a mass change sensor, in accordance with aspects of the present disclosure.

FIG. 3 illustrates a gravity infusion control system in use with an IV administration set including a mass change sensor and a drop counter, in accordance with aspects of the present disclosure.

FIG. 4 is a graph showing calibration curve providing the average drop weight at a specific drop rate.

FIGS. 5A-5C illustrate different infusate drop sizes that resulted from different drop rates.

FIG. 6 is a graph illustrating a calibration curve for normal infusion rate.

FIG. 7 is a graph illustrating the signal to noise ratio of the mass change sensor during keep vein open (KVO) infusion.

FIG. 8 is a graph illustrating a calibration curve for a rapid infusion rate.

FIG. 9 illustrates a potential access point for infusate theft.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV administration set, such embodiments can be used in other fluid conveyance systems. Furthermore, various applications of such embodiments and modifications thereto, which may occur to those who are skilled in the art, are also encompassed by the general concepts described herein.

In accordance with some embodiments, the present disclosure discusses various features and advantages of a gravity infusion control system. The gravity infusion control system can provide for provides numerous improvements for controlling and monitoring medical fluid administration. The gravity infusion control system can monitor and control aspects of medical fluid administration using an IV administration set, and can prevent, detect, and resolve potential or actual complications associated with the medical fluid administration.

The gravity infusion control system includes a user interface, a flow detection interface, and a flow sensing interface. The user interface permits a user to set and/or adjust operational characteristics of the gravity infusion control system, including, but not limited to, dosage and/or volume of medical fluid to be directed to a patient, flow rate of the medicament, start and/or stop parameters for the medical fluid administration, and priming of the fluid pathway for the medical fluid. The user interface, in some embodiments of the present disclosure, can include a graphical user interface.

The means of flow sensing described herein is achieved by two input signals. These signals are the change in mass of the infusate storage container, and the detection of drops falling from a controlled diameter (e.g., drip chamber). Flow sensing is critical to the performance of gravity infusion control systems to ensure the proper rate is achieved and all derived parameters for the user are correct (e.g., estimated time till infusion is complete).

The flow detection interface permits the gravity infusion control system to couple with a medical fluid reservoir or container, and to detect the occurrence of flow from or through the medical fluid reservoir.

The flow detection interface includes an IV administration set coupler that is configured to engage against or retain a portion of an IV administration set forming a medical fluid reservoir, such as an IV bag or drip chamber. The flow detection interface also includes a flow sensor configured to detect any of a change in mass or movement of the medical fluid reservoir to determine the occurrence of flow from or through the medical fluid reservoir. In some embodiments of the present disclosure, the flow sensor can comprise any of a drop sensor, a weight change sensor, and/or an optical sensor.

In some embodiments of the present disclosure, the flow detection interface is configured with one or more IV administration set coupler having a structure for coupling with an IV bag and/or a drip chamber. The structure for the IV bag can be formed as an arm, hook, clamp, or another mechanism configured to suspend the IV bag.

The flow control interface permits the gravity infusion control system to couple with a portion of an IV administration set forming an inner passage for fluid flow, and to control a rate of fluid flow through the inner passage. The flow control interface includes another IV administration set coupler that is configured to engage against or retain a portion of an IV administration set forming an inner passage, such as the IV tubing.

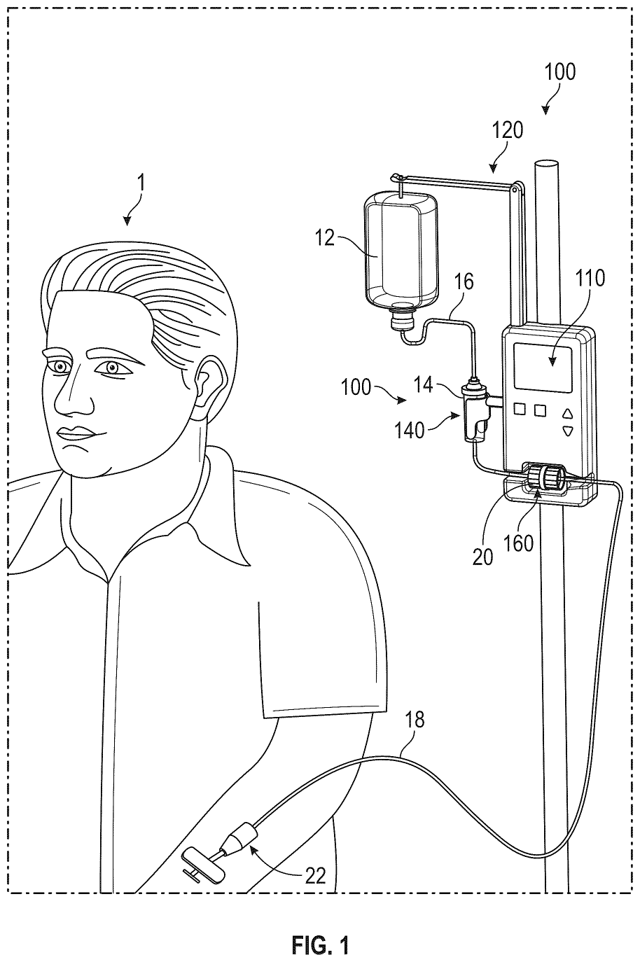

Referring to FIG. 1, an embodiment of a gravity infusion control system 100 is illustrated in use with an IV administration set coupled to a patient 1. The gravity infusion control system 100 includes a user interface 110, a flow detection interface, and a flow control interface. The flow detection interface includes a first IV administration set coupler and a second IV administration set coupler. Further, the flow control interface includes a third IV administration set coupler of the gravity infusion control system.

The gravity infusion control system 100 is coupled with an IV administration set having an IV bag 12 and a drip chamber 14 fluidly coupled together by IV tubing 16 extending therebetween. A flow control device 20 is coupled to a length of IV tubing 18 extending between the drip chamber 14 and the patient 1. The IV tubing is intravenously coupled to the patient through a catheter 22.

The IV administration set and the gravity infusion control system 100 are arranged together with the IV bag 12 coupled to the first IV administration set coupler, the drip chamber 14 coupled to the second IV administration set coupler, and the flow control device 120 coupled to the third IV administration set coupler. After coupling the IV tubing 18 of the IV administration set to the patient, the user interface 110 can be used to start the infusion process. Starting the infusion process can include entering operation parameters, such as a fluid flow rate for the medical fluid and a volume of medical fluid to be administered.

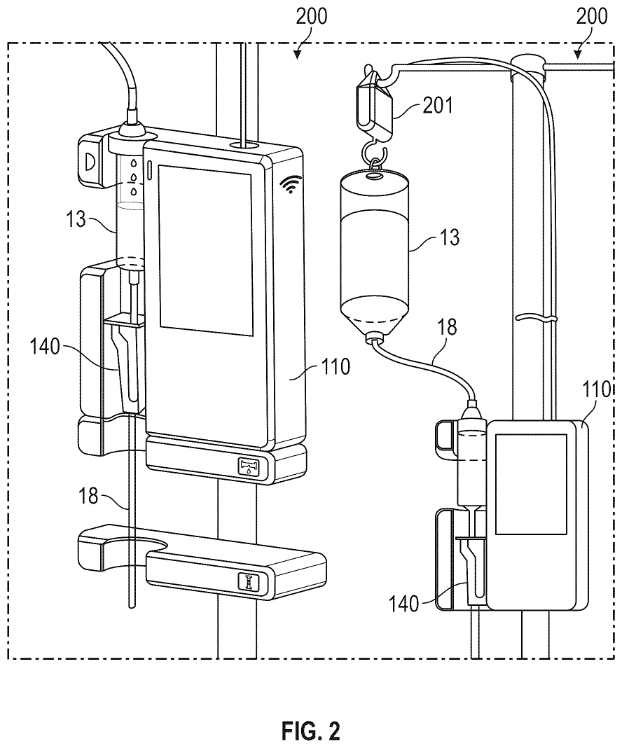

Referring now to FIG. 2, an embodiment of a gravity infusion control system 200 is illustrated, including a mass change sensor 201 as the flow detection interface, a drip chamber 13, a user interface 110, IV tubing 18, and a roller clamp 140. The mass change sensor 201 may be a load cell or a weight, for example. In some embodiments, the mass change sensor 201 is coupled to a drip chamber 13. The structure that couples the mass change sensor 201 to the drip chamber 13 can be formed as an arm, hook, clamp, or another mechanism configured to suspend drip chamber 13.

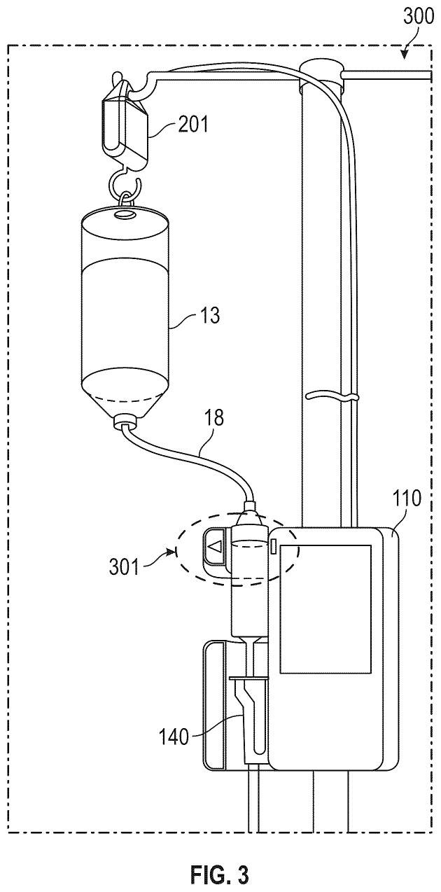

Referring now to FIG. 3, an embodiment of a gravity infusion control system 300 is illustrated, including a mass change sensor 201 as the flow detection interface, a drop detector 301 as the flow sensor, a drip chamber 13, a user interface 110, IV tubing 18, and a roller clamp 140. Gravity infusion control system 300 is similar to gravity infusion control system 200 as described above, with the addition of a drop detector 301. In some embodiments, drop detector 301 is an infrared light emitter and receiver. The mass change sensor 201 may be a load cell or a weight, for example. In some embodiments, the mass change sensor 201 is coupled to a drip chamber 13. The structure that couples the mass change sensor 201 to the drip chamber 13 can be formed as an arm, hook, clamp, or another mechanism configured to suspend drip chamber 13.

The flow sensor can include any sensor to detect movement or a change of weight of the medical fluid reservoir. In some embodiments, the flow sensor can be an optical sensor configured to detect a change of liquid volume in the medical fluid reservoir.

The gravity infusion control system of the present disclosure can further provide features that can increase safety and security related to medication administration. In some embodiments, the gravity infusion control system can provide systems or sub-systems to identify and verify a patient identity. Identification and verification of a patient identity can be conducted using the user interface, a scanner, or another device, such as a smartphone which can communicate with a processor of the gravity infusion control system and/or a patient data system. Identification of the patient can include capturing a photo record of the patient or scanning a barcode on an identification band coupled to the patient.

Additional features of the gravity infusion control system which can increase safety and security related to medication administration can include the capability to identify tampering of the medicament associated with the infusion. The gravity infusion control system can include a weight sensor configured to monitor a weight of the fluid reservoir to also identify tampering of the medical medicament.

The gravity infusion control system of the present disclosure can provide features directed to detecting potential or actual complications related to the infusion process. In some embodiments, the gravity infusion control system can provide systems or sub-systems configured to identify potential complications related to the fluid pathway, including, but not limited to, an occlusion, air in the fluid pathway, a leak or extravasation of the fluid pathway, and instability in the medical fluid flow. To detect potential or actual complications, the gravity infusion control system can include a sensor, such as an air-in-line sensor, a weight sensor, and/or a pressure sensor.

In some embodiments of the present disclosure, the gravity infusion control system is configured to be couplable with one or more sub-system or module, such as an air-in-line subsystem, a tamper-proof subsystem, an infiltration subsystem, a leak detection subsystem, a pressurization subsystem, and/or a patient data subsystem.

The gravity infusion control system can provide a workflow having increased efficiency and reliability, relative to other infusion systems. A workflow associated with the gravity infusion control system can include, but is not limited to a preparation stage, a priming stage, an infusion stage, an infusion maintenance stage, and an infusion conclusion stage. It should be understood that one more stages can occur concurrently or sequentially.

During a preparation stage of the workflow, a caregiver can scan a patient wristband to for identification and verification of the patient. During a priming stage, a caregiver can place an IV administration set in the gravity infusion control system and initiate priming of the fluid pathway. The priming stage may also include indication, by the gravity infusion control system, that there is no complication present, such air in the fluid pathway. During the infusion stage, the caregiver can enter a fluid drop rate and initiate or start the infusion process. The infusion stage, or any other stage, can initiate the saving of documentation or data associated with the infusion process. During an infusion maintenance stage, the gravity infusion control system can provide one or more notification, such as the presence of an occlusion, air-in-line, tampering, leakage, and/or infiltration. Further, during the infusion maintenance stage, the gravity infusion control system can automatically maintain the rate of fluid flow. During an infusion conclusion stage, the gravity infusion control system can automatically clamp or obstruct the fluid pathway of the IV tubing, thereby preventing further medical fluid delivery or unintended leakage of medical fluid during disconnection of the IV administration set from the patient.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1: A gravity infusion control system comprising a drip chamber containing an infusate configured to be fluidly coupled to a patient by IV tubing extending therebetween, a mass change sensor configured to detect any of a change in weight or movement of the infusate in the drip chamber and a drop detector configured to detect drops of the infusate falling from the drip chamber.

Clause 2: The gravity infusion control system of Clause 1 further comprising a user interface that controls a flow rate of the gravity infusion control system.

Clause 3: The gravity infusion control system of Clause 1, wherein the mass change sensor is a load cell or a weight.

Clause 4: The gravity infusion control system of Clause 1, wherein the drip chamber is coupled to the mass change sensor by an arm, a hook, or a clamp.

Clause 5: The gravity infusion control system of Clause 1, wherein an infusate fluid flow is directed through the IV tubing by a pressure of gravity.

Clause 6: The gravity infusion control system of Clause 1, wherein the drop detector is an infrared light emitter and receiver.

Clause 7: The gravity infusion control system of Clause 5, wherein the infusate fluid flow is configured to provide a flow rate about 50 mL/hour, about 100 mL/hour, about 150 mL/hour, about 200 mL/hour, about 250 mL/hour, about 300 mL/hour, about 350 mL/hour, about 400 mL/hour, about 450 mL/hour, or 500 mL/hour.

Clause 8: The gravity infusion control system of Clause 5, wherein the infusate fluid flow is configured to provide a flow rate about 2 mL/hr, about 5 mL/hr, about 10 mL/hr, about 15 mL/hr, about 20 mL/hr, about 25 mL/hr, about 30 mL/hr, about 35 mL/hr, about 40 mL/hr, about 55 mL/hr, or about 5 mL/hr.

Clause 9: The gravity infusion control system of Clause 5, wherein the infusate fluid flow is greater than 500 mL/hr.

Clause 10: The gravity infusion control system of Clause 1 further comprising one or more of an air in line detector, a leak detector, an external pressure module, a tamper resistance module, an information and control module, and a connectivity module.

Clause 11: A gravity infusion control system comprising an intravenous administration set coupled to a medical fluid reservoir, and a flow sensor coupled to the intravenous administration set, the gravity infusion control system configured such that the flow sensor can detect any change in mass or movement of the medical fluid reservoir.

Clause 12: The gravity infusion control system of Clause 11, wherein the flow sensor comprises a mass change sensor and a drop detector.

Clause 13: The gravity infusion control system of Clause 11, wherein the flow sensor is configured to detect any of a change in weight or movement of the medical fluid reservoir of the intravenous administration set.

Clause 14: The gravity infusion control system of Clause 12, wherein the mass change sensor is a load cell or a weight.

Clause 15: The gravity infusion control system of Clause 12, wherein the drop detector is an infrared light emitter and receiver.

Clause 16: A method for providing a gravity infusion control system comprising coupling a medical fluid reservoir of an intravenous administration set to a flow detection interface, wherein the flow detection interface comprises a flow sensor configured to detect any of a change in mass or movement of the medical fluid reservoir, selecting, using a processor, a flow rate through the intravenous administration set, wherein the processor is coupled with the flow detection interface and a flow control interface, such that the processor receives data from the flow sensor to determine the presence of a fluid flow through a tubing of the intravenous administration set.

Clause 17: The method of Clause 14, wherein the flow rate selected is about 50mL/hour, about 100 mL/hour, about 150 mL/hour, about 200 mL/hour, about 250 mL/hour, about 300 mL/hour, about 350 mL/hour, about 400 mL/hour, about 450 mL/hour, or 500 mL/hour.

Clause 18: The method of Clause 14, wherein the flow rate selected is about 2 mL/hr, about 5 mL/hr, about 10 mL/hr, about 15 mL/hr, about 20 mL/hr, about 25 mL/hr, about 30 mL/hr, about 35 mL/hr, about 40 mL/hr, about 55 mL/hr, or about 5 mL/hr.

Clause 19: The method of Clause 14, wherein the flow rate selected is greater than 500 mL/hr.

Clause 20: The method of Clause 14, wherein the wherein the flow sensor comprises a mass change sensor and a drop detector.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim clement is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

### EXAMPLES

### Example 1: Drop Calibration

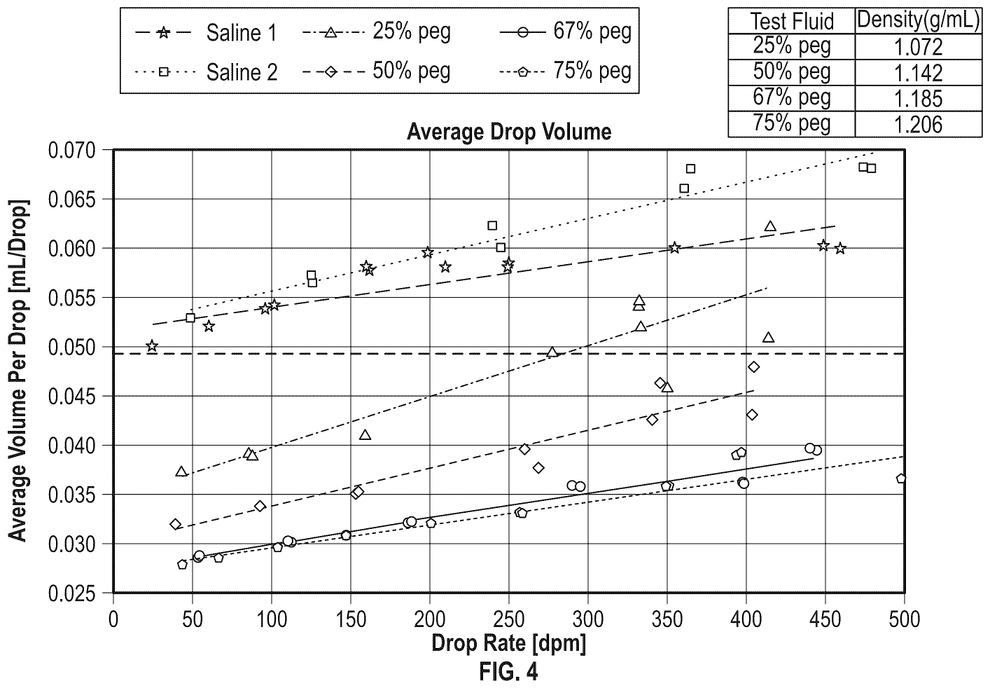

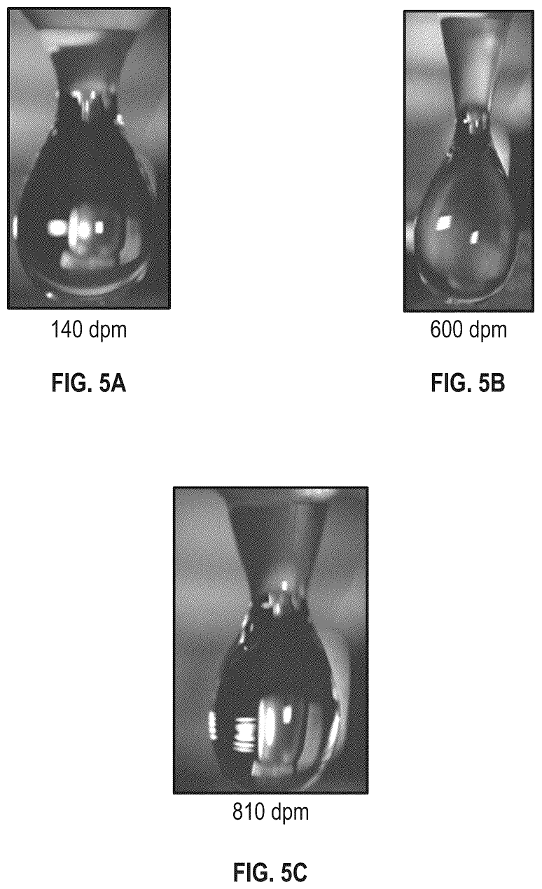

Drop size through a normal drip chamber (e.g., 20 drops/mL) is known to be impacted by the rate of the drops and the fluid properties (e.g., density and surface tension). For this reason, it is desirable if the drop size could be determined for a particular infusate type and at different speeds. The following protocol provides a means of determining the average drop weight of a given infusate at various speeds:
STEP 1: Gravity infusion control system starts from a closed position
STEP 2: Gravity infusion control system opens the flow limiting subsystem to an open rate 1 for a defined period or for a define number of drops. Rate 1 is intended to be a very slow position (˜20 drops per minute). Gravity infusion control system records the drop rate (r1), the number of drops (n1), and the change in mass (dM1)
STEP 3: Gravity infusion control system opens the flow limiting subsystem to an open rate 2 for a defined period or for a defined number of drops. Rate 2 is intended to be a faster position (˜400 drops per minute). Gravity infusion control system records the drop rate (r2), the number of drops (n2), and the change in mass (dM2)
STEP 4: Using the data generated in steps 2 & 3, Gravity infusion control system is able to create a linear calibration curve for the infusate. The calibration curve provides the average drop weight at a specific drop rate. This calibration curve is shown in FIG. 4. Three different drop sizes produced at three different drop rates are shown in FIGS. 5A-5C.

In principle this process could be modified to add as many calibration points as needed.

### Example 2: Density Estimation

Described herein are options for estimating the density of the infusate.
Option 1: For saline like fluids, assume the fluid density is approximately equivalent to 0.9% saline (˜1.0049 g/mL)
Option 2: For saline like fluids, assume the volume of 1 drop at low speeds is 0.05 mL (for a 20 drop per mL drip chamber). Allow a defined number of drops to pass and measure the change in mass (like step 2 or 3 of drop calibration). Density=(Change in mass)/[(number of drops)*(0.05 mL)]
Option 3: Store a library of fluid densities on the gravity infusion control system. When the nurse scans the drug, or enters the drug type, the gravity infusion control system selects the appropriate density value.

### Example 3: Normal Infusion

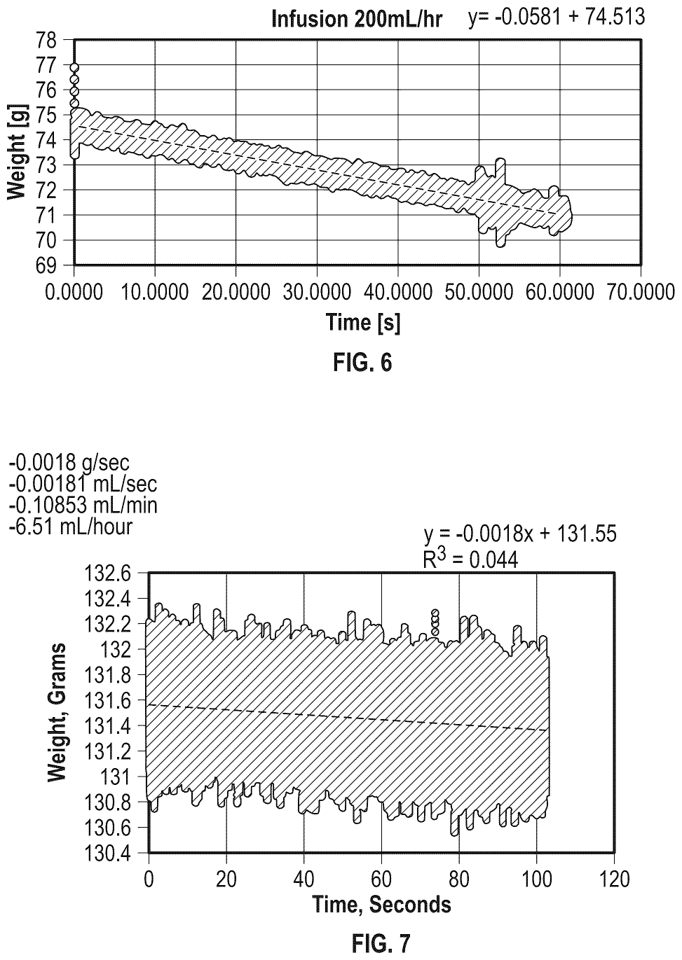

Normal infusion is approximately 50-500 mL/hour. Provided below are two options for sensing the flow rate during normal infusion. FIG. 6 is a graph illustrating an example for a 200 mL/hr infusion rate.
Option 1. Mass Change as Primary: During normal infusion, the flow rate can be sensed from the change in mass over time divided by the assumed or given density of the fluid.
Option 2. Calibrated Drop as Primary: During normal infusion, the flow rate can be sensed from counting the number of falling drops and adjusting its assumed mass based on the linear calibration curve and the assumed or given density of the fluid.

### Example 4: Keep Vein Open (KVO) Infusion

KVO infusion is typically 2-50 mL/hr. In principle KVO infusion control can be similar to what is described for normal infusion except for the following challenges:
1. The time between drops at KVO rates becomes very large (1-2 minutes between drops) making the response time of the system to achieve the correct flow rate very large if it is based on drop rate.
2. The signal to noise ratio of the mass change sensor becomes very large as the change in mass is very small causing noise effects like drift to have a more pronounced effect on the total mass change over time. This is shown in FIG. 7.

To overcome these challenges during KVO infusion, the change in mass signal is used to create the initial rate setpoint since the change in mass is detectable as the drop is forming. This approach results in a fundamentally faster response time than relying solely on the drip counter. Further, the drip counter is to track the total volume over the course of the KVO infusion as the drip counter is less immune to drift and other potential effects than the mass change sensor is.

In this way it is theorized that the two sensors working together allow for the gravity infusion control system to reach the desired flow rate more quickly and have an accurate measurement of the total volume infused over the course of the KVO infusion.

### Example 5: Rapid Infusion

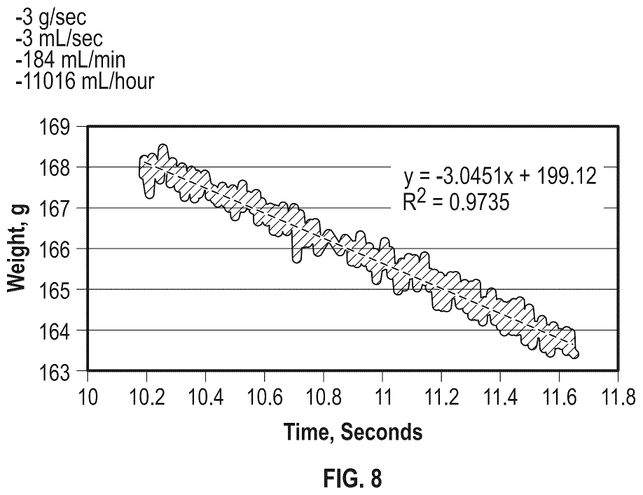

Rapid infusion is typically greater than 500 mL/hr. FIG. 8 is a graph illustrating an example for a rapid infusion rate. In principle, rapid infusion control can be similar to what is described for normal infusion except for the following challenge:

Drops start to coalesce together forming a steady stream, and the drop sensor stops being an effective measurement of volume change over time.

To overcome this challenge during rapid infusion, the mass change sensor is made the primary sensor for measuring flow rate (Option 1 of normal infusion). For these rates, the change in mass will be very high relative to noise effects.

### Example 6: Mechanical Noise on Load Cell

The mass changes sensor is sensitive to events such as: drift, environmental vibrations, shocks or bumps to the IV pole, movement of the IV pole (during transport, or walking), possible line tension (example: during setup the clinician may unintentionally connect the IV set in such a way as to add tension in the line and create a steady external force on the mass change sensor) and other similar events.

If the gravity infusion control system encounters these events, the primary means of detecting flow can temporarily or permanently switch from option 1 (mass change as primary) to option 2 (calibrated drop count as primary).

### Example 7: Infusate Theft or Leak

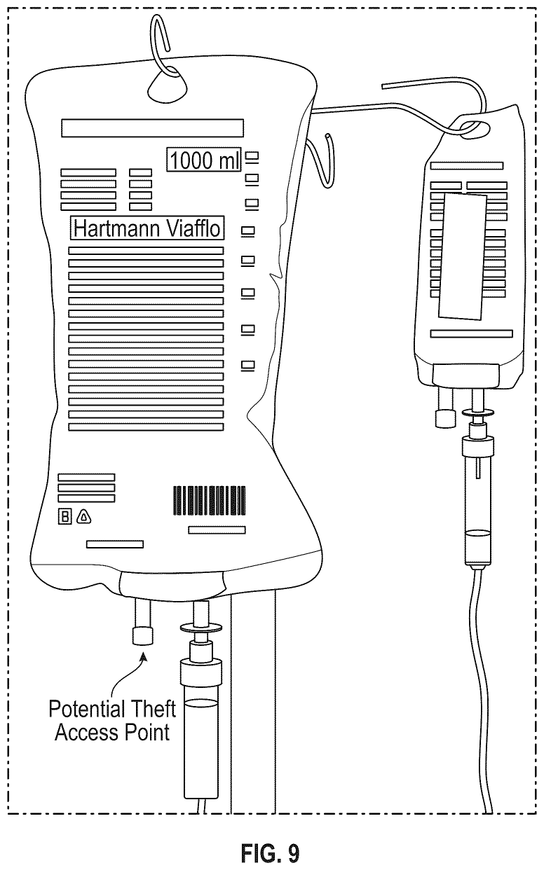

If the flow rate determined from option 1 (mass change as primary) is significantly greater than that determined by option 2 (calibrated drop as primary), it can be surmised that a gross leak or possible infusate theft has occurred. FIG. 9 illustrates a potential access point for infusate theft.

What is claimed is:

1. A gravity infusion control system comprising:
a drip chamber containing an infusate configured to be fluidly coupled to a patient by IV tubing extending therebetween;
a mass change sensor configured to detect any of a change in weight or movement of the infusate in the drip chamber; and
a drop detector configured to detect drops of the infusate falling from the drip chamber.

2. The gravity infusion control system of claim 1 further comprising a user interface that controls a flow rate of the gravity infusion control system.

3. The gravity infusion control system of claim 1, wherein the mass change sensor is a load cell or a weight.

4. The gravity infusion control system of claim 1, wherein the drip chamber is coupled to the mass change sensor by an arm, a hook, or a clamp.

5. The gravity infusion control system of claim 1, wherein an infusate fluid flow is directed through the IV tubing by a pressure of gravity.

6. The gravity infusion control system of claim 1, wherein the drop detector is an infrared light emitter and receiver.

7. The gravity infusion control system of claim 5, wherein the infusate fluid flow is configured to provide a flow rate about 50 mL/hour, about 100 mL/hour, about 150 mL/hour, about 200 mL/hour, about 250 mL/hour, about 300 mL/hour, about 350 ml/hour, about 400 mL/hour, about 450 mL/hour, or 500 mL/hour.

8. The gravity infusion control system of claim 5, wherein the infusate fluid flow is configured to provide a flow rate about 2 mL/hr, about 5 mL/hr, about 10 mL/hr, about 15 ml/hr, about 20 mL/hr, about 25 mL/hr, about 30 mL/hr, about 35 mL/hr, about 40 mL/hr, about 55 mL/hr, or about 5 mL/hr.

9. The gravity infusion control system of claim 5, wherein the infusate fluid flow is greater than 500 mL/hr.

10. The gravity infusion control system of claim 1 further comprising one or more of an air in line detector, a leak detector, an external pressure module, a tamper resistance module, an information and control module, and a connectivity module.

11. A gravity infusion control system comprising:
an intravenous administration set coupled to a medical fluid reservoir;
and a flow sensor coupled to the intravenous administration set, the gravity infusion control system configured such that the flow sensor can detect any change in mass or movement of the medical fluid reservoir.

12. The gravity infusion control system of claim 11, wherein the flow sensor comprises a mass change sensor and a drop detector.

13. The gravity infusion control system of claim 11, wherein the flow sensor is configured to detect any of a change in weight or movement of the medical fluid reservoir of the intravenous administration set.

14. The gravity infusion control system of claim 12, wherein the mass change sensor is a load cell or a weight.

15. The gravity infusion control system of claim 12, wherein the drop detector is an infrared light emitter and receiver.

16. A method for providing a gravity infusion control system comprising:
coupling a medical fluid reservoir of an intravenous administration set to a flow detection interface, wherein the flow detection interface comprises a flow sensor configured to detect any of a change in mass or movement of the medical fluid reservoir;
selecting, using a processor, a flow rate through the intravenous administration set, wherein the processor is coupled with the flow detection interface and a flow control interface, such that the processor receives data from the flow sensor to determine the presence of a fluid flow through a tubing of the intravenous administration set.

17. The method of claim 14, wherein the flow rate selected is about 50 mL/hour, about 100 mL/hour, about 150 mL/hour, about 200 mL/hour, about 250 mL/hour, about 300 mL/hour, about 350 mL/hour, about 400 mL/hour, about 450 mL/hour, or 500 mL/hour.

18. The method of claim 14, wherein the flow rate selected is about 2 mL/hr, about 5 mL/hr, about 10 ml/hr, about 15 mL/hr, about 20 mL/hr, about 25 ml/hr, about 30 mL/hr, about 35 mL/hr, about 40 mL/hr, about 55 mL/hr, or about 5 mL/hr.

19. The method of claim 14, wherein the flow rate selected is greater than 500 mL/hr.

20. The method of claim 14, wherein the wherein the flow sensor comprises a mass change sensor and a drop detector.
